(19) United States
(12) Patent Application Publication
(10) Pub. No.: US 2020/0191767 A1
(43) Pub. Date: Jun. 18, 2020

(54) Controlling Translocating Molecules Through A Nanopore

(71) Applicants: THE UNIVERSITY OF OTTAWA, Ottawa (CA); University of Rochester Medical Centre, Rochester, NY (US)

(72) Inventors: Vincent TABARD-COSSA, Québec (CA); Kyle BRIGGS, Ottawa (CA); Gregory MADEJSKI, Albion, NY (US); James L. MCGRATH, Fairport, NY (US)

(21) Appl. No.: 16/609,099

(22) PCT Filed: Apr. 27, 2018
(86) PCT No.: PCT/US2018/029939

§ 371 Date: Oct. 28, 2019

Related U.S. Application Data
(60) Provisional application No. 62/491,572, filed on Apr. 28, 2017.

(51) Int. Cl. G01N 33/487; C12Q 1/6869

(52) U.S. Cl. CPC G01N 33/48721; C12Q 2565/631; C12Q 1/6869

(57) ABSTRACT

To reduce unwanted variation in the speed of DNA translocating solid-state nanopores, a nanoscale pre-confinement of translocating molecules is demonstrated using an ultra-thin nanoporous silicon nitride (NPN) membrane separated from a single sensing nanopore by a nanoscale cavity. Comprehensive experimental results demonstrate that the presence of this nanofilter results in a global minimum in the coefficient of variation of passage times in the sensing pore over a range of DNA sizes which depends on the height of the cavity. Such advanced nanopore devices minimize the standard deviation of the passage time distribution independently of its diameter and stability. These results also represents the first experimental verification that the inter- and intra-molecular passage time variation depends on the conformational entropy of such molecule prior to translocation, while providing a practical strategy for controlling transport across solid-state nanopores.

### CROSS-REFERENCE TO RELATED APPLICATIONS

This application claims the benefit of U.S. Provisional Application No. 62/491,572, filed on Apr. 28, 2017. The entire disclosure of the above application is incorporated herein by reference.

### FIELD

The present disclosure relates to a nanopore device and method for making the same.

### BACKGROUND

When a single biopolymer, such as DNA, translocates a nanopore, the dynamics of molecular transport are complex. The speed during passage is thought to be dependent on the fraction and conformation of the molecule outside the pore, as well as being subject to thermal fluctuations and transient interactions with the pore walls and membrane materials. The net effect is for the molecular motion to be afflicted by a wide distribution of passage speeds, both due to inter- and intra-molecular velocity fluctuations. Such spread in passage times confounds simple translation of time to molecular position, complicating mapping applications, and greatly limits the ability of the nanopore to distinguish charged molecules by size compared to traditional gel electrophoresis techniques.

While years of intensive research are now bearing fruit in the form of sequencing devices based on biological nanopores, in part due to the high level of motion control achieved through the use of enzymes to ratchet DNA through the pore, significant challenges remain in leveraging the potential offered by solid-state nanopores.

Most experimental efforts relating to translocation speed control have focused on slowing DNA by various means, including laser-modulating surface charge density to control electroosmotic flow; by a judicious choice of electrolyte, both aqueous and ionic-liquid; by adjustment of the viscosity; by interfacing the pore with a gel; or by using different membrane materials. While these methods are able to slow DNA translocations to varying degrees, they do so at the cost of broader passage time distributions.

Only a few studies have considered the factors that contribute to the wide distributions of passage times. The choice of salt solution has been shown to have a significant effect on the width of passage time distributions. Simulation work has demonstrated that polymers are perturbed from equilibrium by the extended electric field gradient, broadening the passage time distribution compared to equilibrium predictions. Molecules which are extended prior to translocation have longer passage times due to increased drag forces, and speed up toward the end of the translocation.

Unfortunately, while the dominant mechanism responsible for high variability in passage time is thought to be the large conformational entropy available to DNA molecules prior to translocation through the nanopore, experimental verification has remained difficult due to the complexity of fabricating devices with sufficiently confining geometries in the vicinity of a nanopore.

This section provides background information related to the present disclosure which is not necessarily prior art.

### SUMMARY

This section provides a general summary of the disclosure, and is not a comprehensive disclosure of its full scope or all of its features.

A system is presented for controlling translocation of a target molecule through a nanopore. The system includes: a sensing structure, two chambers configured to host a fluid and fluidly coupled to each other by a fluidic channel, such that the sensing structure is disposed into fluidic channel and thereby prevents the fluid from passing between the two chambers except through the nanopores formed therein; and two electrodes electrically coupled to a voltage source, where the electrodes are configured to apply an electrical potential across the sensing structure, such that one electrode is placed into each of the two chambers.

A sensing membrane is deposited onto a substrate and the sensing membrane includes a single nanopore formed therein. The sensing membrane may be comprised of a dielectric material or a two-dimensional material. One or more spacers are disposed onto an exposed top surface of the sensing membrane. A filter membrane is disposed over the one or more spacers and onto the top surface of the sensing membrane, whereby the sensing membrane, the one or more spacers and the filter membrane form a sensing structure. The filter membrane includes a plurality of nanopores formed.

In some embodiments, the filter membrane is separated from the sensing membrane by a distance on the order of contour length of the target molecule. For example, the one or more spacers are configured to separate the filter membrane from the sensing membrane by a distance and sized such that the distance is less than contour length of the target molecule.

In some embodiments, the average size of a nanopore in the plurality of nanopores formed in the filter membrane is less than twice the radius of gyration of the target molecule.

In some embodiments, the filter membrane is configured to exhibit electrical resistance lower than the electrical resistance exhibited by the sensing membrane.

In some embodiments, the filter membrane and the sensing membrane define a space between them such that volume of the space between the filter membrane and the sensing membrane is less than a thousand times the volume of the target molecule when the target molecule is in coiled form in a free solution.

In some embodiments, the plurality of nanopores are formed in the filter membrane with an average nearest neighbor distance between any two nanopores such that two times the distance between the filter membrane and the sensing membrane plus the average nearest neighbor distance between any two nanopores is greater than contour length of the target molecule.

In some embodiments, the plurality of nanopores are formed in the filter membrane with an average nearest neighbor distance between any two nanopores such that the average nearest neighbor distance between any two nanopores is less that radius of gyration of the target molecule when the target molecule is in a free solution.

In some embodiments, the filter membrane is configured to exhibit electrical resistance and the sensing membrane is configured to exhibit an electrical resistance such that quotient of the electrical resistance exhibited by the filter membrane divided by the electrical resistance exhibited by the sensing membrane is less than 0.01.

In one aspect, the sensing structure can be used to control translocation of a target polymer through a sensing membrane having a single nanopore formed therein. The method includes: positioning a sensing structure in a fluidic channel; driving a target polymer through the nanopore in the sensing structure by applying an electric potential across the sensing structure; and measuring passage time of the target polymer through the nanopore in the sensing membrane, where a distance separating the filter membrane from the sensing membrane is less than contour length of the target molecule.

In another aspect, the method includes: positioning the sensing structure in a fluidic channel; driving a target polymer through the nanopore in the sensing structure by applying an electric potential across the sensing structure; and measuring passage time of the target polymer through the nanopore in the sensing membrane, where the plurality of nanopores formed in the filter membrane with an average nearest neighbor distance between any two nanopores such that the average nearest neighbor distance between any two nanopores is less that radius of gyration of the target molecule when the target molecule is in a free solution.

In yet another aspect, the method includes: driving a target polymer through the sensing nanopore in the sensing structure and into a cavity formed between the filter membrane and the sensing membrane, where the target polymer is driven by applying an electric potential across the sensing structure; trapping the target polymer in the cavity, where distance separating the filter membrane from the sensing membrane is larger than capture radius of the target polymer; and ejecting the target polymer from the cavity in the sensing structure by reversing the electric potential applied across the sensing structure.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a side view of an improved sensing structure for use in a system for translocating a molecule through a nanopore;

FIG. 2 is a top view depicting a spacer with holes arranged on the sensing membrane;

FIGS. 3A-3C are side views depicting the transfer of the filter membrane onto the sensing membrane, a gap being maintained between them by means of the spacer;

FIG. 4 is a diagram of an example setup for a nanopore device;

FIGS. 5A and 5B are exploded view of the support structure for the nanopore device;

FIG. 6 is a cross-sectional side view of the support structure along with zoomed in view of the sensing structure therein;

FIGS. 7A-7C illustrate different length polymers passing through the sensing structure ad FIGS. 7D-7F are corresponding graphs showing passage time distributions for the respective polymers;

FIG. 8 is a graph showing that polymers which are very long with respect to the dimensions of the nanodevice will clog the device by threading through the sensing pore while straddling two or more pores in the filter membrane.

FIGS. 9A and 9B are graphs showing the mean passage times for the proposed nanodevice and a control device, respectively;

FIGS. 10A and 10B are graphs showing the standard deviation of passage times for the proposed nanodevice and a control device, respectively;

FIGS. 11A and 11B are graphs showing the coefficient of variation of passage times for the proposed nanodevice and a control device, respectively;

FIGS. 12A-12C are graphs showing a 500-bp DNA ladder in a pore (12A and 12B) for the proposed nanodevice and a control device (12C);

FIG. 13 is a graph showing the fold fraction for various nanodevices in relation to a control device; and

FIGS. 14A and 14B are graphs showing proof-of-principle data for operation of the proposed nanodevice as an entropic trap.

Corresponding reference numerals indicate corresponding parts throughout the several views of the drawings.

### DETAILED DESCRIPTION

Example embodiments will now be described more fully with reference to the accompanying drawings.

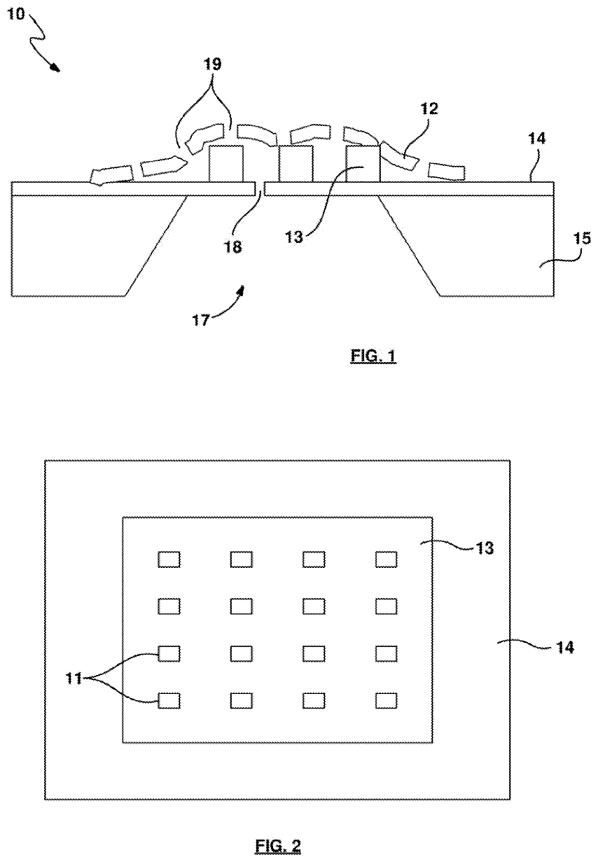

FIG. 1 depicts an improved sensing structure 10 for use in a system for translocating a molecule through a nanopore. The sensing structure 10 is comprised generally of a filter membrane 12 disposed onto an exposed top surface of a sensing membrane 14. One or more spacers 13 are preferably disposed onto the top surface of the sensing membrane 14 and thereby separate the filter membrane 12 from the sensing membrane 14. The distance separating the filter membrane from the sensing membrane is preferably on the order of the contour length of the target molecule (e.g., a monomer or a polymer). The one or more spacers 13 help to form a cavity between the filter membrane 12 and the sensing membrane 14. This cavity has a volume that is typically less than a thousand times the volume of the target molecule when the target molecule is in a coiled form in a free solution. For structural support, the membranes are deposited onto a substrate 15.

In an example embodiment, the sensing membrane 14 is preferably comprised of a dielectric material, such as silicon nitride. A thin film of silicon nitride (e.g., 5-100 nm) may be deposited onto the substrate (e.g., a silicon wafer) using low-pressure chemical vapour deposition (LPCVD). The sensing membrane 14 may also be comprised of other dielectric or non-dielectric materials including a multilayered metallic/dielectric construction or a two-dimensional material such as graphene or a transition metal dichalcogenide.

Spacers 13 are formed on the exposed top surface of the sensing membrane 14. In the example embodiment, a spacer 13 is formed by placing a layer of silicon dioxide (e.g., 50-1000 nm) onto the silicon nitride using plasma enhanced CVD (PECVD) or other deposition techniques. A photoresist is then patterned on the silicon dioxide layer and selectively etched away (e.g. using dry RIE or wet HF) to form one or more holes (i.e., a grid) in the spacer 13 on the sensing membrane 14 which serves to maintain separation between the sensing membrane 14 and the filter membrane 12. In the example embodiment, the spacer 13 is a film with an array of holes 11 formed therein as best seen in FIG. 2. In another embodiment, the spacers may be comprised of an array of pillars formed on the top surface of the sensing membrane. Other arrangements for the spacers and their fabrication are contemplated by this disclosure.

A recess 17 is also formed on the opposing side of the substrate to expose the sensing membrane. Likewise, a photoresist may be patterned onto the substrate and then etched to form the recess 17 in the substrate 15. In the example embodiment, the recess 17 aligns with a center of the spacer 13 arranged on the opposite side of the wafer. It is readily understood that the recess 17 may be formed before or after the formation of the spacer 13 but is preferably prior to the filter membrane 12 being transferred onto the sensing membrane 14.

The filter membrane 12 is also comprised of a dielectric material, such as silicon nitride. In the example embodiment, the filter membrane 12 is a thin film (e.g., on the order of 1 to 100 nanometers) which may be supported on a substrate 15. The filter membrane 12 includes a plurality of nanopores 19 with tunable hole diameters ranging from 1-100 nanometers (also referred to herein as nanofilter pores). Preferably, the average size of the nanofilter pores is less than twice the radius of gyration of the target molecule. In one embodiment, an example filter membrane 12 is commercially available from SiMPore, Inc. in West Henrietta, N.Y. The fabrication of such filter membranes is further described in PCT patent application no. PCT/US2014/051316 which is incorporated herein in its entirety. In other embodiments, the filter membrane 12 is fabricated using gas-phase sequential infiltration synthesis or other atomic layer deposition methods. Other fabrication method for the filter membrane are also contemplated by this disclosure.

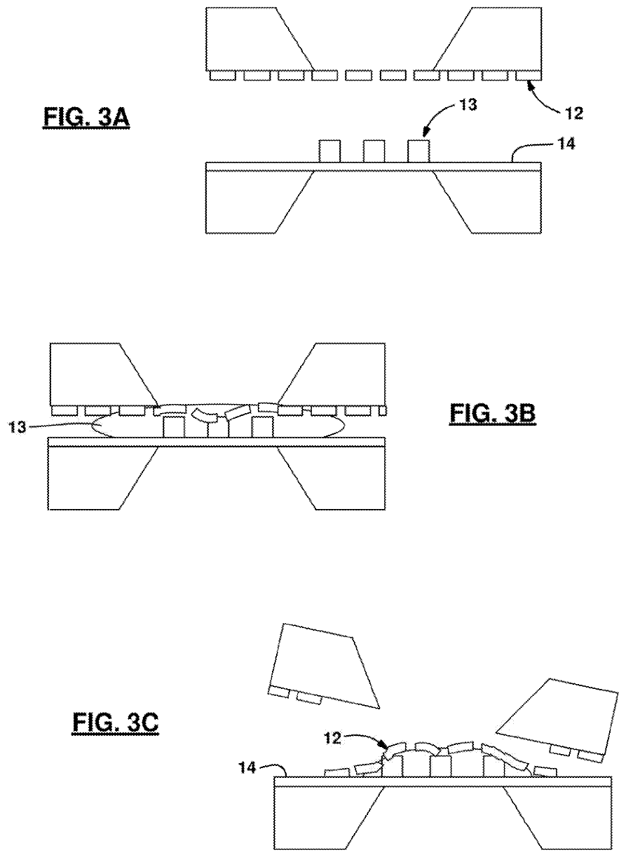

By bringing the filter membrane 12 in close contact with the sensing membrane 14, the filter membrane 12 can be deposited over the spacer 13 and onto the sensing membrane 14. In one example, Van Deer Waal's force may be used to transfer the filter membrane 12 onto the sensing membrane 14. Referring to FIGS. 3A-3B, the membranes are brought into close proximity to each other (e.g., a few microns) as seen in FIG. 3A. In some case, the membranes may be brought into contact with each other. Each component may be cleaned (e.g., by corona treatment) to make the surface hydrophilic.

In FIG. 3B, a stream of cool water vapour 31 is directed at the filter membrane 12 which inundates the cavity between the two membranes. The water between the membranes is allowed to evaporate. As it does, surface tension causes the two membranes to be pulled together, such that they stick together once contact is made between the membranes. Lastly, the substrate supporting the filter membrane 12 is removed mechanically. The filter membrane 12 tears away from the substrate 15 and remains behind on top of the spacer 13 as seen in FIG. 3C. To complete the sensing structure, the filter membrane 12 may be affixed to the sensing membrane 14, for example by applying polydimethylsiloxane along the edges of the filter membrane 12 and thereby sealing it in place. The newly formed sensing structure 10 may also be cured by baking at 80 degrees Celsius.

Alternatively, the filter membrane 12 can be transferred using a stream of compressed gas (e.g. nitrogen or compressed air) directed at the filter membrane 12 which causes it to deflect toward the spacer 13 and make contact such that the two membrane stick together (e.g. due to Van der Waal's forces) once contact is made between the membranes. To facilitate the bonding of the two materials, their surfaces can be activated by air or oxygen plasma prior to contact. In addition, while compressed by a jig, the sensing structure 10 can be thermally annealed at high temperature to thermally bond the filter membrane 12 to the spacer 13, replacing the need for applying polydimethylsiloxane or some other adhesive and thereby avoid having to tear the substrate away of the filter membrane. While references have been made to particular transfer methods, other ways of depositing the filter membrane onto the sensing membrane are contemplated by this disclosure.

In some embodiments, the sensing membrane 14 includes a sensing pore 18 that is formed during the fabrication of the sensing structure 10. For example, the sensing pore may be formed by electron- or ion-beam drilling after the silicon nitride has been deposited on the substrate. In other embodiments, the sensing pore 18 in the sensing membrane 14 may be formed using a controlled breakdown process in the context of a nanodevice as will be further described below. Other techniques for forming the sensing pore are also envisioned. Likewise, it is readily understood that other fabrication methods may be used to form the sensing structure 10.

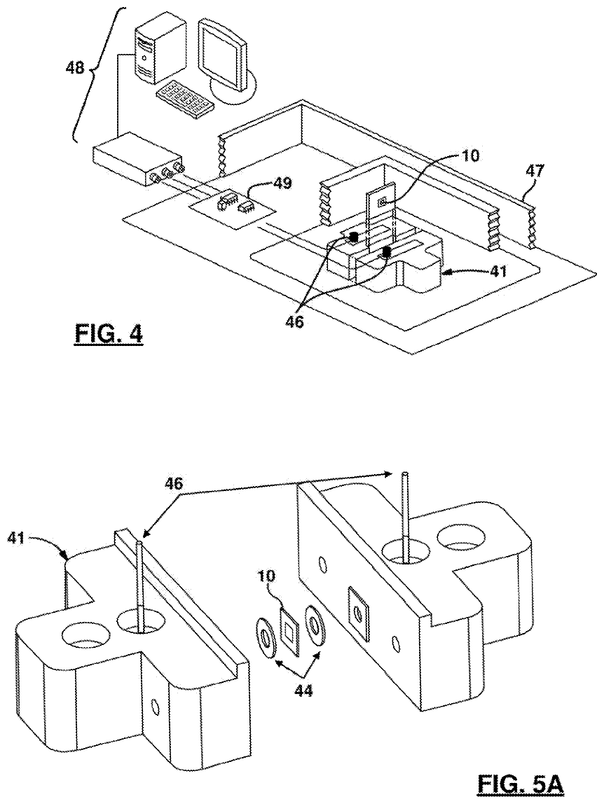

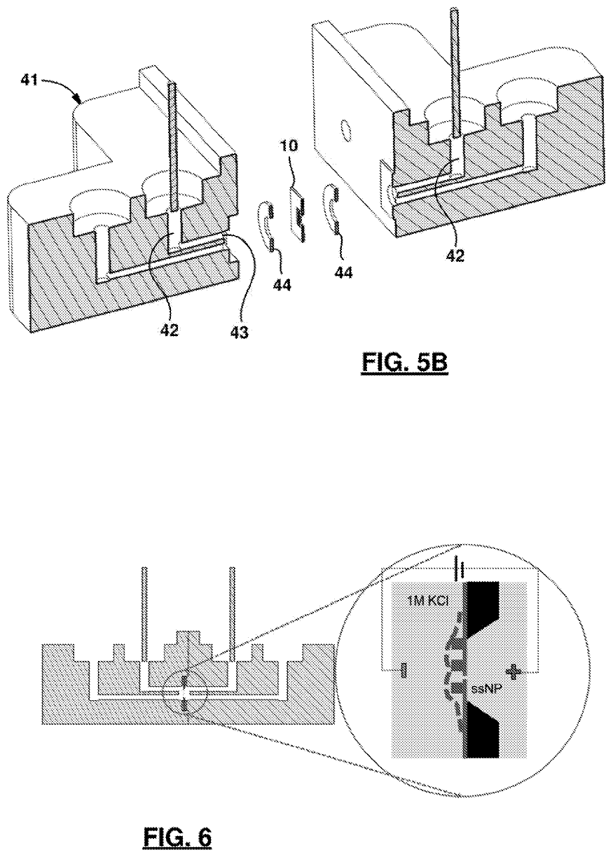

FIGS. 4-6 illustrate an example setup for a nanopore device 40. The nanodevice 40 is comprised primarily of a support structure 41 which defines various fluidic passageways therein. In this example, the support structure 41 includes at least two chambers 42 fluidly coupled to each other by a fluidic channel 43. The two chambers 42 and the fluidic channel 43 host a fluid containing ions. In one example, the fluid is potassium chloride dissolved in water. In another example, the fluid is a non-aqueous solvent, such as lithium chloride in ethanol. Depending on the analyte of interest, it is readily understood that the composition of the fluid may vary.

The sensing structure 10 is disposed into the fluidic channel 43 and thereby prevents fluid from passing between the two chambers except through the nanopores formed in the sensing structure 10. In this example, the support structure 41 is formed by two separate pieces which mate together. The sensing structure 10 fits into a recess formed in one or both of the two separating pieces. The sensing structure 10 may be sandwiched between two silicone gaskets 44. Other configurations for hosting the sensing structure 10 by the support structure 41 as well as for the supporting structure 41 itself are envisioned by this disclosure.

Two electrodes 46 are inserted into respective chambers 43 of the nanodevice. The electrodes are electrically coupled to a voltage source 49 and configured to apply an electrical potential across the sensing structure 10 disposed in the nanodevice 40. The voltage source 49 is controlled by a controller 48. In one embodiment, the controller is implemented by a data acquisition circuit electrically coupled to a personal computer or another type of computing device. In some embodiments, the data acquisition circuit may also be configured to measure the current flowing between the two chambers of the support structure. In other embodiments, the supporting structure 41 and/or the entire system can be disposed in a grounded faraday cage 47 to isolate electric noise. Thus, the setup is similar to what is commonly used for biomolecular detection in the nanopore sensing field.

As noted above, the sensing pore may be formed in the sensing membrane using a controlled breakdown process. In this case, an electric potential is applied across the sensing membrane while the sensing structure 10 is disposed in the support structure 41 of the nanopore device 40, such that the electric potential that induces an electric field having a value greater than 0.1 volt per nanometer across the sensing membrane. Through a controlled breakdown of the material comprising the sensing membrane, a single nanopore is formed in the sensing membrane. While the electric potential is being applied across the sensing membrane, leakage current is monitored across the sensing membrane. Formation of the nanopore is determined by an abrupt increase in the leakage current across the sensing membrane. In response to detecting the abrupt increase in leakage current, the electric potential across the membrane is removed. Further details regarding the controlled breakdown process may be found in U.S. Patent Publication No. 2015/0108008 which is incorporated by reference in its entirety.

Different applications are envisioned for the nanodevice described above. For example, a mixture of an analyte, such as DNA or other charged molecules, may be flowed from one chamber through the sensing structure 10 to the other chamber of the nanodevice 40. For most applications, the analyte will enter on the side of the filter membrane 12 although in some applications (i.e., entropic cages) the analyte may enter on the side of the sensing membrane 14. In operation, a small voltage (e.g., up to one volt) drives the analyte through the system by electrophoresis.

In an example embodiment, dimensions of the nanodevice and its sensing structure may be tailored to its application. Dimensions for the nanodevice are defined as:

h: vertical distance between the sensing membrane and the nanofilter

x: average nearest-neighbor distance between any two pores in the nanofilter

L: the contour length of the polymer being studied

Y: diameter of the holes in the spacer material

Short polymers are viewed as any polymer with L<h. Short polymers must fully enter the gap before being captured by the sensing pore and will partially relax in so doing. While the nanodevice still passes these polymers, it has little effect on the translocation kinetics through the sensing pore.

Medium polymers are viewed as polymers satisfying h<L<3(x/2+h). Medium polymers represent the length range over which the nanodevice is most useful for sizing applications. Here, polymers generally do not clog the sensing pore while threading two nanofilter pores, but the translocation kinetics are strongly affected by the filter membrane.

Long polymers are viewed as any polymer satisfying L>3(x/2+h). Long polymers will sooner or later clog the sensing pore by having both ends enter it while threading two nanofilter pores, getting stuck when a constant electric field is applied. Note that because only a few nanofilter pores are actually active, the actual nearest-neighbour pore distance x may be different from the average for a given device, so the ranges for the polymer lengths given above are just estimates that will work most of the time. Some devices may have a slightly different value for the transition between medium and long polymers, but on average this will work reasonably well. Y is constrained only in that it must be small enough to be able to prevent the flexible nanofilter layer from touching the sensing membrane, though one could envision decreasing Y in order better focus the electric field lines in the cavity and thereby achieve a larger electric field gradient with which to stretch polymers

In sum, the nanodevices work best to elongate polymers without clogging when polymers are of medium length as defined above. Dimensions of the nanodevice should be tuned so that the polymer of interest is in the medium range.

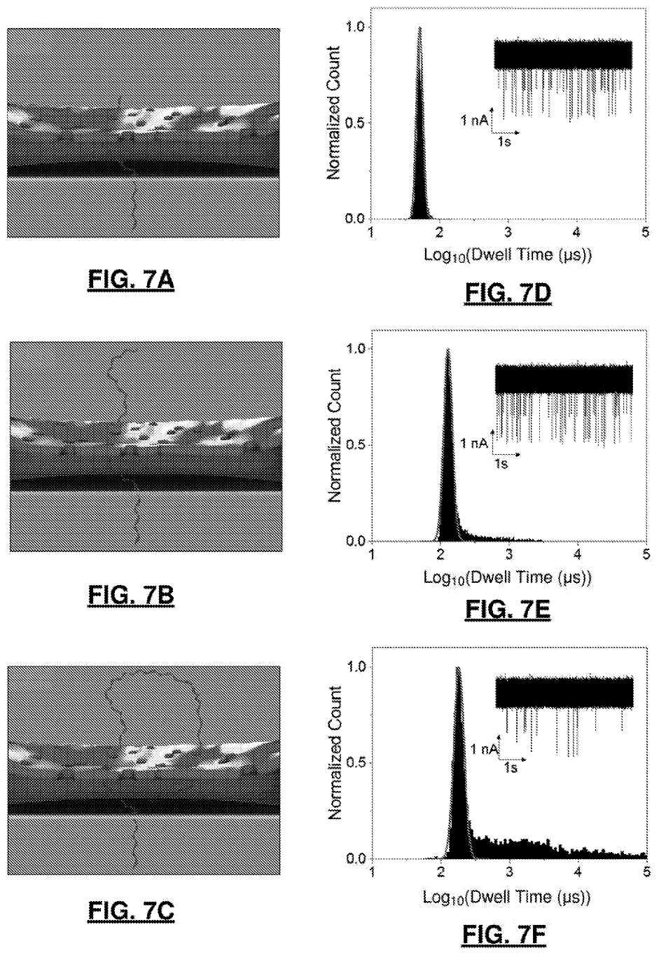

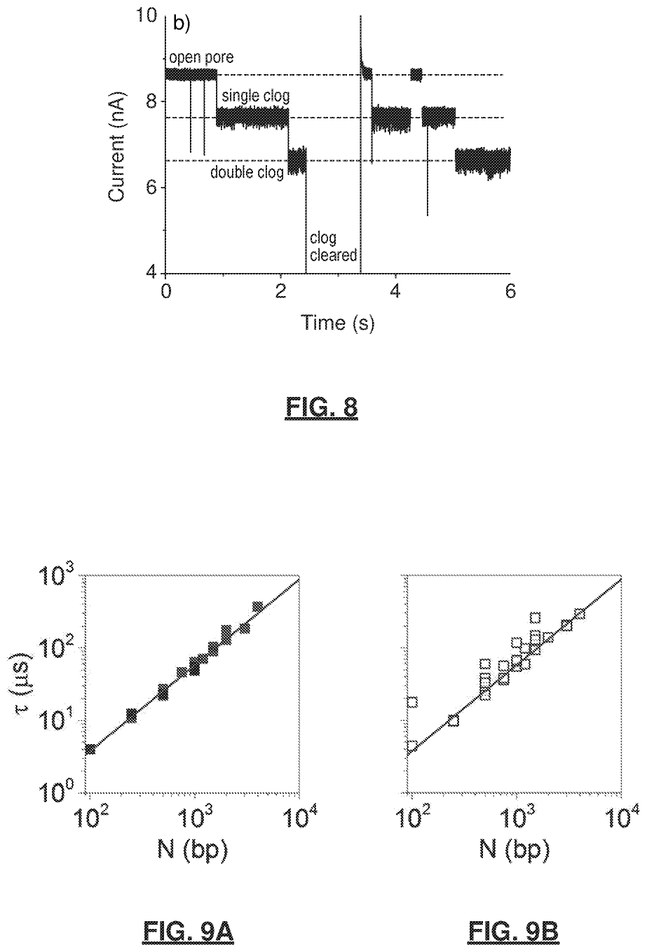

In one aspect of this disclosure, the passage time distribution characteristics are controlled by properly configuring the nanodevice 40 for a particular application. Short polymers are defined herein as a polymer which is too short to be threaded through two nanofilter pores 19 while being captured by the sensing pore. With reference to FIGS. 7A and 7D, short polymers show approximately log-normal passage time distributions. As the polymer length increases, a tail of longer passage times emerge as seen in FIGS. 7B and 7E. In FIGS. 7C and 7F, longer polymers are threaded through two nanofilter pores 19 while being captured by the sensing pore 18. These longer polymers can not only thread through two nanofilter pores, can have a part of their length lying flat along the nanofilter membrane, increasing the likelihood of friction and sticking, greatly increasing the size of the long passage time tail in the distribution. Polymers which are long enough to have both ends in the sensing pore at the same time while being threaded through two or more pores in the nanofilter exhibit predictable clogging modes in which both ends of the polymer can get captured by the sensing pore while threading through two nanofilter pores, thereby clogging the sensor. This sets an upper limit on the length of the polymer which can be reliably studied with the nanodevice 40 for a given spacer geometry.

A method for improving translocation of a target polymer through a sensing membrane of the nanodevice 40 is set forth. As a starting point, the sensing structure is positioned in the fluidic channel of the nanodevice as described above. A target polymer is driven through the nanopore in the sensing structure by applying an electric potential across the sensing structure. The passage time of the target polymer through the nanopore in the sensing membrane is then measured. Of note, the one or more spacers are configured to separate the filter membrane from the sensing membrane and sized such that the distance is less than contour length of the target molecule. In some instances, the plurality of nanopores formed in the filter membrane are also configured to ensure that the polymer is unlikely to clog. For example, dimensions for the filter membrane may be desgined as follows

$$h + \frac{x}{2} > \frac{L}{3} .$$

This is a generous bound and assumes some degree of relaxation. A firmer bound would be 2h+x>L. That is, the plurality of nanopores in the filter membrane have an average nearest neighbor distance between any two nanopores such that two times the distance between the filter membrane and the sensing membrane plus the average nearest neighbor distance between any two nanopores is greater than contour length of the target molecule.

For polymers in the range that can be usefully studied with the nanodevice 40, the filter membrane 12 suppresses the standard deviation of the passage time, allowing maximal resolution in the passage time distributions as seen in FIGS. 9-11. The origin of this effect is in the stretching and elongation of DNA as it passes through the filter membrane. As it does so, it must uncoil and elongate in order to pass through the filter, since the nanofilter pores are too small for it to pass in a coiled configuration. Once one end is in the space between the two membranes, an electric field gradient is present, which means that the leading end (closest to the sensing pore) of the polymer will experience a stronger pulling force than the rest of the polymer. This mechanism of stretching also happens without the filter membrane. However, coupled with the friction from contact with the filter membrane, the polymer will stretch more, reducing the number of possible conformations that the polymer can take on prior to capture. Because the polymer must linearize and stretch itself in order to pass through the filter, the filter membrane limits the number of conformations that the polymer can assume as it passes, leading to highly consistent translocation kinetics every time.

While the electric field gradient is still present in control devices, with a physical extent that scales as the second power of the diameter of the sensing pore, the elongation is less complete than when the filter membrane is not present, particularly for small pores. The filter membrane thus eliminates the dependence on sensing pore size and its size stability, making all pores, even small ones, very consistent sensors of passage of analytes of interest. At its heart, the filter membrane is a mechanism by which to maximally stretch polymers as they approach the sensing pore.

In the example embodiment, the pores in the filter membrane are randomly distributed. Due to the fact that nanofilter pores are randomly distributed, the lengths of polymer which delimit the regimes discussed above can vary slightly between devices depending on the local distribution of nanofilter pores near the sensing pore. Nonetheless, it is possible to predict where the regimes will fall on average. In other embodiments, the pattern and location of the pores in the filter membrane can be controlled such that one can also predict where the regimes will fall.

In other instances, clogging can be used to extend the time that a polymer spends in the sensing pore. For this application, dimensions of the nanodevice should be tuned so that the polymer of interest is in the long range. A clogged polymer could be controllably moved, for example by superimposing an AC electric field over a DC field while clogged. The polymer's extended lifetime in the pore would allow for more accurate mapping of features along the polymer length and/or the use of lower bandwidth electronics for detection purposes.

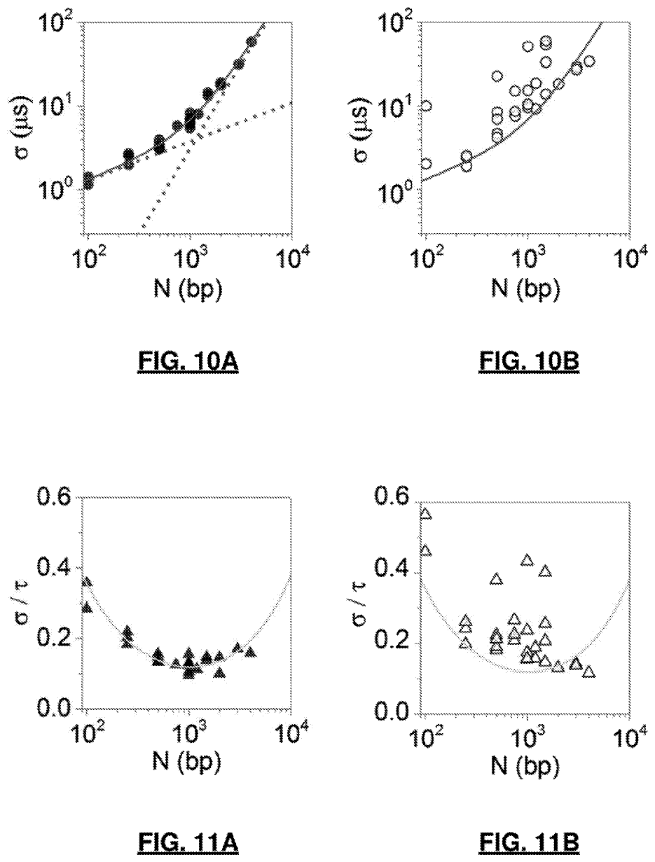

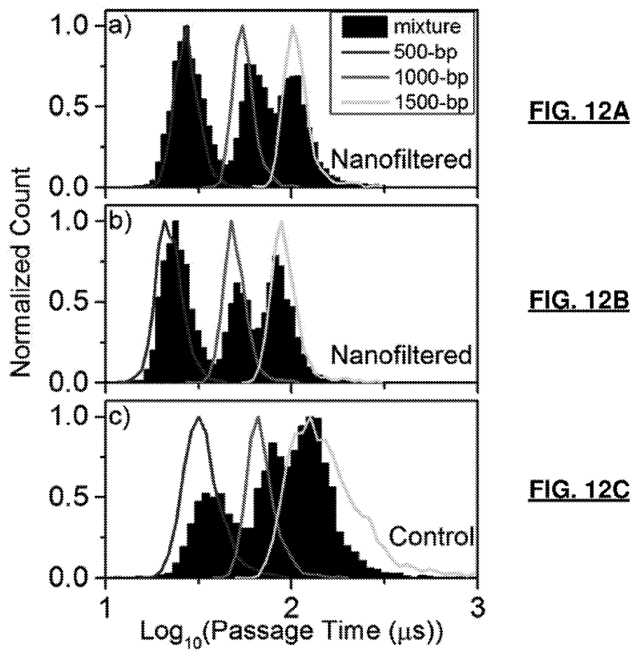

Nanopore size spectroscopy is another application for the nanodevice 40. Because the standard deviation of the passage time is minimal when using a nanofilter (see, FIGS. 9 and 10), this can be leveraged to improve the DNA size resolution for distinguishing mixed populations of DNA fragments. The critical improvement here is that while regular nanopores previously could only achieve about 1000-bp resolution in distinguishing DNA by length, or they could achieve ˜400-bp resolution (specifically, distinguish 100-bp from 500-bp) if the pore was precisely the right size, the proposed nanodevice 40 can achieve 500-bp resolution in the short to intermediate polymer length range using any sensing pore size, even if the sensing pore is unstable in a LiCl salt. Better performances (e.g. ˜100 bp) are expected using different cation species to shield the DNA charge, such as K⁺, in a KCl solution for example. Without the filter membrane, the distributions depend critically on the sensing pore size, which makes devices unreliable and failure-prone, while with the filter membrane in place this dependence is gone, and size spectroscopy becomes more reliable.

In another aspect of this disclosure, the proposed sensing structure can also be configured to suppress folded translocation of molecules. In this aspect, the sensing structure is again positioned in the fluidic channel of the nanodevice as described above. A target polymer is driven through the nanopore in the sensing structure by applying an electric potential across the sensing structure and the passage time of the target polymer through the nanopore in the sensing membrane is measured. In this case, the average nearest neighbor distance between any two nanopores is configured in a particular manner to suppress folding. Specifically, the plurality of nanopores are formed in the filter membrane with an average nearest neighbor distance between any two nanopores such that the average nearest neighbor distance between any two nanopores is less that radius of gyration of the target molecule when the target molecule is in a free solution.

Experimental data validates this approach to suppressing folding. Define type 1 events to be single-file passage of DNA, type 2 events to be events in which the DNA is folded in the middle and translocates entirely folded, and type 21 events to be partially folded events in which the event begins in the folded state. Fold fraction is defined as

$$f = \frac{{t}_{2}}{{t}_{total} + {t}_{2}} ,$$

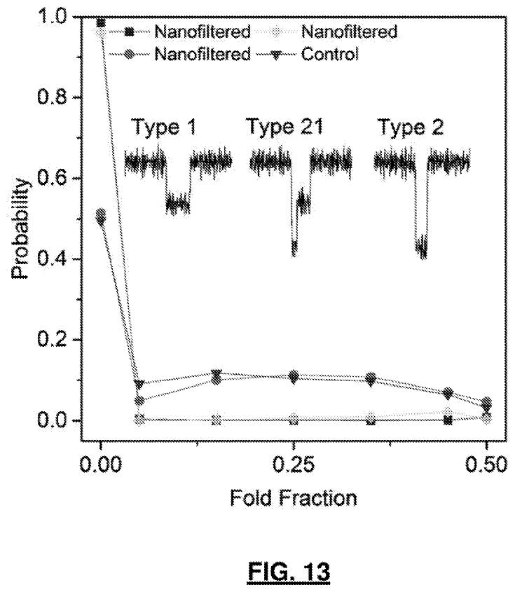

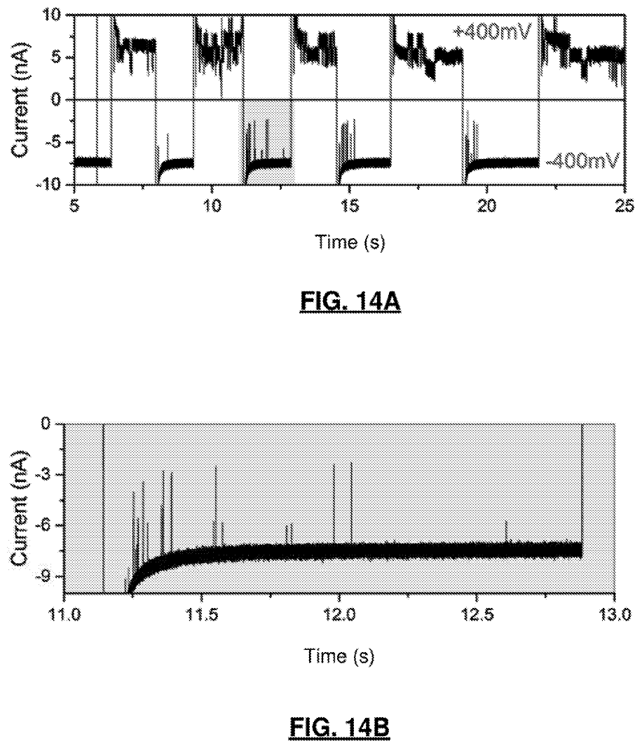

the ratio of time spent in the folded state t₂ to the sum of the total passage time and the folded time tₜₒₜₐₗ+t₂. This can be used as proxy for the fractional location of the fold along the DNA backbone. Type 1 events have f=0, type 2 events have f=0.5 and partially folded type 21 events fall in between. Examples of each type can be seen in the inset to FIG. 13.

With continued reference to FIG. 13, it is noted that two of the eight nanodevices studied nearly completely suppressed folded translocations, despite the sensing pore being large enough to allow folded passage. However, the remaining six nanodevices showed behavior similar to the control devices (i.e., without a filter membrane). Since folding suppression happened (or not) for all DNA lengths studied with a particular device, it must be a static property of the filter membrane which is responsible for folding suppression, but which is not necessarily present in every nanodevice most likely because of the randomly distributed size and location of the pores in the nanofilter.

Because DNA is a stiff polymer, it requires a significant force to fold into a nanopore. Because of the low impedance of the filter membrane, the vast majority of the voltage drops across the sensing pore, and only a very small voltage drop, and therefore a small force is present at the filter membrane. Because the electric field decays away from the sensing pore as the square of the radial distance from the sensing pore, only the nanofilter pores which are within a lateral distance of about the gap height away from the sensing pore actually have enough voltage drop across them to allow passage of DNA. For the porosities considered, that means fewer than eight nanofilter pores are actively passing DNA. If all of the nanofilter pores that are close to the sensing pore are very small (i.e., defined as being smaller than the persistence length of double-stranded DNA or about 30 nm), then the force will be insufficient to allow DNA to pass the filter in a folded configuration. Because the time the DNA spends in between the membranes is insufficient to fully relax, unfolded passage at the filter will predispose unfolded passage at the sensing pore. In this case, the filter membrane is optimized by having small (<30 nm) nanofilter pores.

Alternatively, if the two nanofilter pores nearest the sensing pore happen to be very close together, such that any DNA molecule passing through the nanofilter almost always gets caught by both pores at the same time from either end, then it will have to linearize and choose a nanofilter pore to pass through before it can actually translocate (i.e., a situation like that depicted in FIG. 7C). This process will tend to linearize the DNA and promote unfolded capture since the polymer will be linearized by competing pulling forces prior to translocating the sensing pore. In this case, the filter membrane is optimized by having a very high porosity such that the average distance between pores was smaller than the intermembrane gap height, enabling folding suppression even though the nanofilter pores are not necessarily smaller than the persistence length of the polymer.

In this scenario, for folding to be suppressed requires that x<<L (e.g., x<L/5), so that the polymer is very likely to thread through two nanofilter pores, and h>L/2 is required, so that the polymers are not so long as to be able to get both ends into the sensing pore at the same time. Again, Y is constrained only in that it must be small enough to be able to prevent the flexible filter membrane from touching the sensing membrane. In order to suppress folding of 1000 bp dsDNA (340 nm) for example, one would need x<68 nm and h>170 nm. Y=1000 nm works here as well for the nanofilter characteristics used (50 nm thick). Other dimensions can be constrained as described above.

Another way of controlling translocation of a target polymer is into and out a cavity formed between the filter membrane and the sensing membrane of the sensing structure. A target polymer may be driven into the cavity by applying an electric potential across the sensing structure. For example, if the nanodevice 40 is run in reverse, so that DNA is captured from the sensing pore side and gets pulled into the intermembrane space, it functions as an entropic trap. Because the force on the DNA is small inside the gap between the nanofilter and the sensing membrane, DNA will tend to be trapped there longer than if there were no nanofilter in place. Thus, the target polymer is trapped in the cavity. In order to maximize this trapping time, it is important to use a large intermembrane gap h, at least as large as the capture radius of the sensing pore (usually on the order of 100 to 1000 nanometer). This ensures that the voltage drop across the nanofilter is insufficient to overcome diffusion, and should result in very long trapping times. The capture radius is difficult to estimate well, but generally is on the order of 100 to 1000 nm, so one can say the requirement is h>1000 nm and d<30 nm for dsDNA. Subsequently, the target polymer is ejected from the cavity by reversing the electric potential applied across the sensing structure. The difference with this device is that it can operate on any length of polymer that can fit into the gap.

Additional tuning parameters to maximize the trapping time are to use very small nanofilter pores (e.g., <30 nm) which will present a larger entropic barrier to translocation. Increasing the porosity will also reduce the electric field at the nanofilter and thereby make escape more difficult. In general, there is an interplay between nanofilter pore size and porosity, and these parameters must be tuned together to maximize trapping time. The parameter which will be most important for trapping applications will be the ratio of the electric resistance of the active area of the nanofilter membrane to that of the sensing pore; this value should be very small for entropic trapping applications. For example, the ratio of the electrical resistance exhibited by the filter membrane to the electrical resistance exhibited by the sensing membrane is preferably less than 0.01.

FIG. 14 shows proof of principle data for the operation of these devices as entropic traps, showing loading and subsequent recovery of trapped molecules. The nanocages could find application as nanoscale reactors, in which DNA is loaded into the trap via the sensing pore, where it interacts with some small molecules which are present on the nanofilter side of the system and which are sufficiently small to diffuse freely through the nanofilter and into the intermembrane space. The voltage can then be reversed to recapture and study the reaction products.

Another possible application is a concentrator, where a low-abundance sample is captured in the inter-membrane gap at high voltage to produce a locally high concentration inside the gap, before reversing and reducing the magnitude of the voltage and recapturing at low voltage to study in more detail.

The filter membrane serves generally as a filter. These nanofilters have a very sharp size exclusion for globular molecules while allowing linear polymers to pass through even if their free solution size is larger than the nanofilter pores. This can be used as a filter to clean up a real-world sample (e.g. blood) which will contain a larger number of different kinds of biomolecules which would otherwise clog the sensing pore. One could tune the size of the nanofilter pores in order to exclude all background molecules while allowing passage of only targets of interest. All that is required here is that the nanofilter pores be small enough to exclude the smallest particle that one needs filtered (d<f), where f is diameter of smallest particle that needs to be filtered, and that the target polymers under study are of short to medium length. Thus, dimensions of the nanopore filters will vary depending upon the application and the size of expected molecules in the sample.

Because the fluid in the gap between the nanofilter and the sensing pore is necessarily quiescent and free from flow, the nanofilter can be used to create a shear-free region of fluid in contact with the sensing pore even when lateral flow exists above the nanofilter membrane. This enables a high capture rate of polymers by the sensing pore, since there is no shear flow occurring at the sensing pore location, while also allowing flow to be used above the nanofilter in order to facilitate bringing new analyte to the vicinity of the pore.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” “disposed onto”, or “coupled to” another element or layer, it may be directly on, engaged, connected to, disposed onto, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A system for controlling translocation of a target molecule through a nanopore, comprising:
a sensing membrane deposited onto a substrate, wherein the sensing membrane includes a single nanopore formed therein;
one or more spacers disposed onto an exposed top surface of the sensing membrane;
a filter membrane disposed over the one or more spacers and onto the top surface of the sensing membrane, whereby the sensing membrane, the one or more spacers and the filter membrane form a sensing structure and wherein the filter membrane includes a plurality of nanopores formed therein;
two chambers configured to host a fluid and fluidly coupled to each other by a fluidic channel, wherein the sensing structure is disposed into fluidic channel and thereby prevents the fluid from passing between the two chambers except through the nanopores formed therein; and
two electrodes electrically coupled to a voltage source and configured to apply an electrical potential across the sensing structure, such that one electrode is placed into each of the two chambers.

2. The system of claim 1 wherein the filter membrane is separated from the sensing membrane by a distance on the order of contour length of the target molecule.

3. The system of claim 1 wherein average size of a nanopore in the plurality of nanopores formed in the filter membrane is less than twice the radius of gyration of the target molecule.

4. The system of claim 1 wherein the filter membrane is configured to exhibit electrical resistance lower than the electrical resistance exhibited by the sensing membrane.

5. The system of claim 1 wherein filter membrane and the sensing membrane define a space between them such that volume of the space between the filter membrane and the sensing membrane is less than a thousand times the volume of the target molecule when the target molecule is in coiled form in a free solution.

6. The system of claim 1 wherein the sensing membrane is comprised on a dielectric material.

7. The system of claim 1 wherein the sensing membrane is comprised on a two-dimensional material.

8. The system of claim 1 wherein the one or more spacers are configured to separate the filter membrane from the sensing membrane by a distance and sized such that the distance is less than contour length of the target molecule.

9. The system of claim 8 wherein the plurality of nanopores formed in the filter membrane with an average nearest neighbor distance between any two nanopores such that two times the distance between the filter membrane and the sensing membrane plus the average nearest neighbor distance between any two nanopores is greater than contour length of the target molecule.

10. The system of claim 1 wherein the plurality of nanopores formed in the filter membrane with an average nearest neighbor distance between any two nanopores such that the average nearest neighbor distance between any two nanopores is less that radius of gyration of the target molecule when the target molecule is in a free solution.

11. The system of claim 1 wherein the filter membrane is configured to exhibit electrical resistance and the sensing membrane is configured to exhibit an electrical resistance such that quotient of the electrical resistance exhibited by the filter membrane divided by the electrical resistance exhibited by the sensing membrane is less than 0.01.

12. A sensing structure for controlling translocation of a target molecule through a nanopore, comprising:
a sensing membrane with a single nanopore formed therein;
one or more spacers disposed onto a surface of the sensing membrane; and
a filter membrane disposed over the one or more spacers and onto the surface of the sensing membrane, wherein the filter membrane includes a plurality of nanopores formed therein and the one or spacers are sized such that the filter membrane is separated from the sensing membrane by a distance on the order of contour length of the target molecule.

13. The sensing structure of claim 12 wherein the filter membrane is configured to exhibit electrical resistance lower than the electrical resistance exhibited by the sensing membrane.

14. The sensing structure of claim 12 wherein filter membrane and the sensing membrane define a space between them such that volume of the space between the filter membrane and the sensing membrane is less than a thousand times less the volume of the target molecule when the target molecule is in coiled form in a free solution.

15. The sensing structure of claim 12 wherein the plurality of nanopores formed in the filter membrane with an average nearest neighbor distance between any two nanopores such that two times the distance between the filter membrane and the sensing membrane plus the average nearest neighbor distance between any two nanopores is greater than contour length of the target molecule.

16. The sensing structure of claim 12 wherein the plurality of nanopores formed in the filter membrane with an average nearest neighbor distance between any two nanopores such that the average nearest neighbor distance between any two nanopores is less that radius of gyration of the target molecule when the target molecule is in a free solution.

17. The sensing structure of claim 12 wherein the filter membrane is configured to exhibit electrical resistance and the sensing membrane is configured to exhibit an electrical resistance such that quotient of the electrical resistance exhibited by the filter membrane divided by the electrical resistance exhibited by the sensing membrane is less than 0.01.

18. A method for controlling translocation of a target polymer through a sensing membrane having a single nanopore formed therein, comprising:
positioning a sensing structure in a fluidic channel, the sensing structure having a filter membrane disposed onto a sensing membrane, where the filter membrane includes a plurality of nanopores formed therein and the filter membrane is separated by one or more spacers from the sensing membrane;
driving a target polymer through the nanopore in the sensing structure by applying an electric potential across the sensing structure; and
measuring passage time of the target polymer through the nanopore in the sensing membrane, where a distance separating the filter membrane from the sensing membrane is less than contour length of the target molecule.

19. The method of claim 18 wherein plurality of nanopores in the filter membrane are formed with an average nearest neighbor distance between any two nanopores such that two times the distance between the filter membrane and the sensing membrane plus the average nearest neighbor distance between any two nanopores is greater than contour length of the target molecule.

20. A method for controlling translocation of a target polymer through a sensing membrane having a single nanopore formed therein, comprising:
positioning a sensing structure in a fluidic channel, the sensing structure having a filter membrane disposed onto a sensing membrane, where the filter membrane includes a plurality of nanopores formed therein and the filter membrane is separated by one or more spacers from the sensing membrane;
driving a target polymer through the nanopore in the sensing structure by applying an electric potential across the sensing structure; and
measuring passage time of the target polymer through the nanopore in the sensing membrane, where the plurality of nanopores formed in the filter membrane with an average nearest neighbor distance between any two nanopores such that the average nearest neighbor distance between any two nanopores is less that radius of gyration of the target molecule when the target molecule is in a free solution.

21. A method for controlling translocation of a target polymer into a sensing structure of a nanodevice, where the sensing structure includes a filter membrane having a plurality of nanopores and a sensing membrane having a single sensing nanopore formed therein, comprising:
driving a target polymer through the sensing nanopore in the sensing structure and into a cavity formed between the filter membrane and the sensing membrane, where the target polymer is driven by applying an electric potential across the sensing structure;
trapping the target polymer in the cavity, where distance separating the filter membrane from the sensing membrane is larger than capture radius of the target polymer; and
ejecting the target polymer from the cavity in the sensing structure by reversing the electric potential applied across the sensing structure.
